(19) United States
(12) Patent Application Publication
(10) Pub. No.: US 2017/0196729 A1
(43) Pub. Date: Jul. 13, 2017

(54) Method to Draw Heat out Of a Living Item Caused by Burns and / or Destructive inflammation That Causes Heat

(71) Applicant: Mark Lelong, Twin Peaks, CA (US)

(72) Inventor: Mark Lelong, Twin Peaks, CA (US)

(21) Appl. No.: 15/285,402

(22) Filed: Oct. 4, 2016

Related U.S. Application Data
(60) Provisional application No. 62/200,718, filed on Aug. 4, 2015.

(51) Int. Cl. A61F 7/00; A61F 7/02

(52) U.S. Cl. CPC A61F 7/00; A61F 7/02; A61F 2007/0002; A61F 2007/0029; A61F 2007/0039; A61F 2007/0021

(57) ABSTRACT

The current invention uses prior technology commonly known as a heatsink as indicated above. The current invention uses a dome or dish style heat source that when applied against a living heat source it will draw heat out of the living item and dissipate it into the air cooking the living item. The new item added to the current invention will allow for a way of handling/holding the current invention while isolating transfer of heat that could affect the thermodynamic efficacy of the current invention without it.

### CROSS-REFERENCE TO RELATED APPLICATIONS

Provisional application 62/200/718 filed: Aug. 4, 2015

I'm seeking the benefit of the provisional application restored by filing a grantable petition (including a statement that the delay in filing the nonprovisional application was unintentional and the required petition fee) to restore the benefit under 37 CFR 1.78—Statement to be included

### STATEMENT REGARDING FEDERALLY SPONSORED RESEARCH OR DEVELOPMENT

This invention was not federally sponsored research or development—Mark LeLong

### BACKGROUND OF THE INVENTION

Conventional use of a “heatsink” or “Heat Sink” has been around for a long time. Commonly they are made of materials that dissipate heat quickly. The principle of use is for one side to be permanently fastened by screws or other clamping device to a heat source, with the opposing side slotted to create fins, in a manner that as cool air moves thru the fins a thermodynamic imbalance is created causing the heat to be drawn away from the heat source towards the cool air in the fins letting the air dissipate the heat from the fins.

Conventional use of a “heatsink” or “Heat Sink” are commonly related to mechanical or electric heat sources, where as a new use and improvements by additional use and additional components will make this an improvement on an existing “heatsink” or “Heat Sink” use.

The present invention is in the technical field of Medical, More particularly the present invention is in the technical field of burn and heat inflammation treatments caused by and/or influenced by internally hosted and/or external elements

Conventional devices and or solutions, such as Lotions, creams and/or sprays are typically used on the skin to provide comfort. These products are usually designed from a mixture of chemicals be it man made of natural. The embodiments of these chemicals are applied wet, prohibiting the use on or near an open wound.

Conventional treatments offer a form of relief by penetrating the skin by way of absorption, thus any relief is subject to the elapsed time needed to absorb such treatments.

Conventional treatments do not offer any method to remove heat from such burns or inflammation. They will hide, mask or deaden the sensation of burns and inflammation, but they do nothing to remove the physical heat

Conventional treatments do not offer any method to remove heat from such burns or inflammation that originate below the skin surface and only affects the skin as an unintentional target to reach a deeper treatment site. This might happen commonly in the treatment of cancer by the use of Radiation beam therapy systems of all types.

Conventional treatments might be the use of an ice bag or a bag full of some other form of frozen media; however the freezing cold temperatures of these Items can prove damaging if applied for an extended time frame. As these items return to their normal temperature they can become moist and sweat offering liquid that can enter an open wound

While these common heatsinks may be suitable for the purposes for which they were designed, they would not be as suitable for the purposes of the present invention, as hereinafter described.

### SUMMARY OF THE PRESENT INVENTION

The present invention discloses a new use for an existing patentable item commonly known as a “Heatsink” or “Heat Sink” in a common commercially available embodiment, plus with the additional components described in this application this invention will also provide additional new uses for this commonly known item “heatsink” or “Heat Sink”.

A primary object of the present invention is to provide a heatsink invention that may remove heat from a living entity

Another object of the present invention is to provide a heatsink invention that may remove heat from a living entity upon contact without the need to be mounted or secured in a permanent manner like other heatsinks using screw and or other hardware to remove heat from a living entity

Another object of the present invention is to provide a heatsink invention that may have a domed or dish surface to make contact with a living entity for heat removal, unlike common heatsinks that have a flat machined contact surface that would prove ineffective on a living entity's surface.

Another object of the present invention is to provide a heatsink invention that may have a thermodynamic design ratio of (3 to 1 or higher as described in detail below) to remove heat from a living entity

Another object of the present invention is to provide a heatsink invention that may have a handle that allows the present invention to be held and/or manipulated in a manner that insolates it from absorbing heat from the entity holding it

Another object of the present invention is to provide a heatsink invention that may have the ability to be used singularly or as multiple units that when joined by perforations or punctures in a sheet of flexible material, the present invention can be used to remove heat from a living entity that is more like a tube shape like a leg, arm or scalp to list just a small potential items not to be consider the exclusive option or use for this and should be consider more of a guide line.

Additional objects of the present invention will appear as the description proceeds.

The foregoing and other objects and advantages will appear from the description to follow. In the description reference is made to the accompanying drawings, which form a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying drawings, like reference characters designate the same or similar parts throughout the several views.

The following detailed description is, therefore, not to be taken in a limiting sense, and the scope of the present invention is best defined by the appended claims.

### BRIEF DESCRIPTION OF THE DRAWINGS

In order that the invention may be more fully understood, it will now be described, by way of example, with reference to the accompanying drawings in which:

FIG. 1 is a visual representation of a basic heatsink embodiment used in many industries commercially. The current invention will describe and visually show new structural modifications that provide for new uses and addition of components to further provide new uses for a prior patent.

FIG. 2 is a side view of the present invention. Shown is the current invention with the heat source item #1 being created in the form of a dome as its heat source surface, unlike the prior item in FIG. 1 The Item in FIG. 1. Shows to be machined flat making this dome design unique and new with a use that is unobtainable with the design in FIG. 1.

FIG. 3 is a side view of the present invention. Shown is the current invention with the heat source item #1 being created in the form of a dish as its heat source surface, unlike the prior item in FIG. 1 The Item in FIG. 1. Shows to be machined flat making this dish design unique and new with a use that is unobtainable with the design in FIG. 1.

FIG. 4 is a top view of the present invention. Shown is the current invention with the fins machined into the top of the current invention. As items #1 & #2 represents a machined lip/ledge that will allow the item of FIG. 5. To rest against, when pushed over the fins to create a handle as a new addition to the invention in FIG. 1.

FIG. 5 is a multiple view of the Additional new item that provides a new use for the present invention. FIG. 5. Also shows the drastic changes needed to the heatsink in FIG. 1. For it to use this new use, however as heatsinks by design physic require the use of a non-furious metal the addition to the present invention could also give a new use to an existing invention with appropriate machining and/or casting

1. Top view of the new handle addition

2. Side view of the new handle addition

3. Item 4 is a cross section of Items 1 & 3 as if cut at 3.2

4. Is a cross section of the item showing a chamfer at 4.5 providing for comfort when held as a handle.

FIG. 6 is a top view of the present invention. Shown is the current invention with multiple copies of the current invention that are bound into a single assembly similar to the example in this drawing, however the size, quantity of the current invention and shape is and should be assumed to vary to a particular need of use. The new addition to the use and additional functionality of the current invention comes from the sheet of a plastic material of a flexible nature Item #1 that allows the above describe unit to wrap around an arch or a tube type shape like an arm or leg as an example but not to imply the only use on an angle of arch or tube.

FIG. 7 is a side view of the present invention. Shown is the current invention with multiple copies of the present invention combined in a sheet of insolating pliable material that might typically be of a plastics family being applied to a arch or tube shaped item similar to an arm or leg but by no count should be considered the only use as this is a general description of use but not the sole totality.

### DESCRIPTION OF THE PREFERRED EMBODIMENT(S)

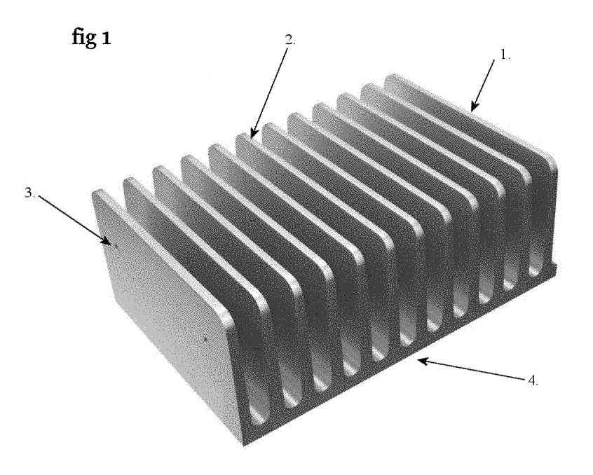

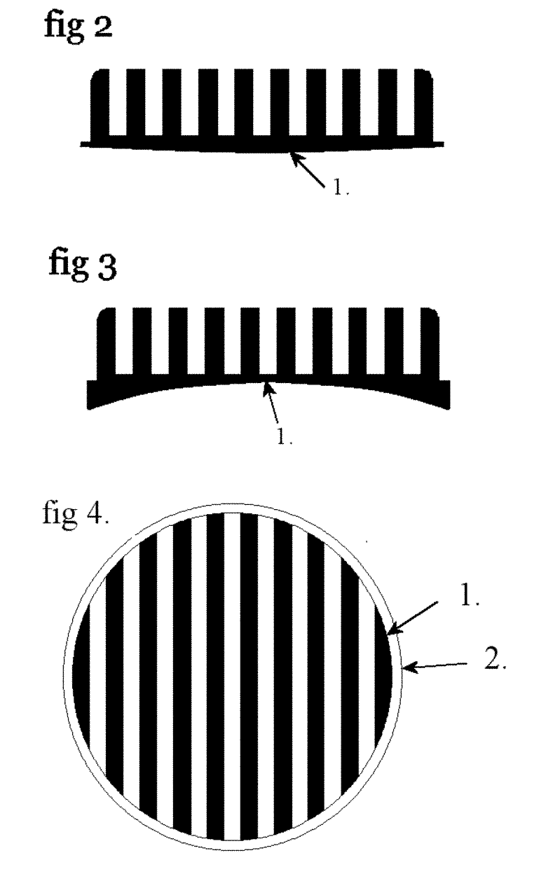

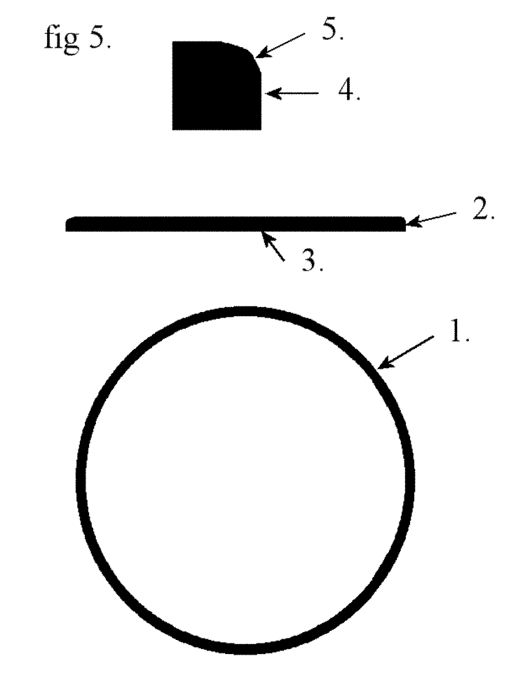

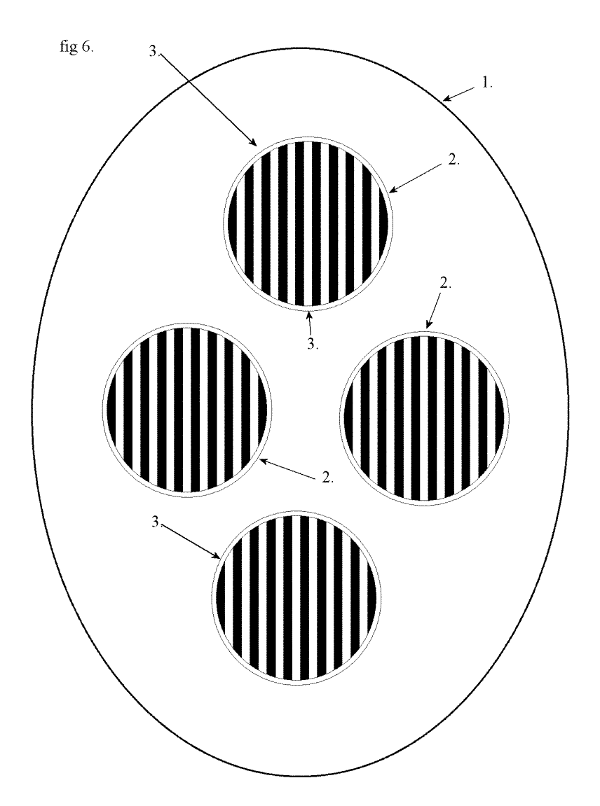

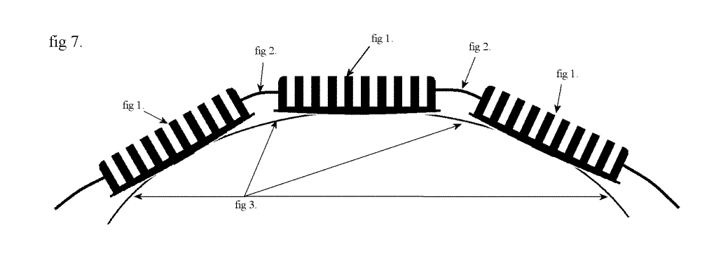

In order that the invention may be more fully understood, it will now be described, by way of example, with reference to the accompanying drawings in which FIGS. 1 through 8 illustrate the present invention being an heatsink designed to remove heat from a living entity and/or the removal of heat caused by inflammation of tissue internal or external to a living entity.

This invention relies on prior inventions patentable features and claims. While this invention relies on these prior claims, it also will describe new uses and additions that will make it patentable. Conventional claims and uses for this type of invention might be known as a “heatsink” or “Heat Sink” FIG. (1.1). For this description I will use Heatsink as the common name and spelling as reference only and not to exclude those inventions that are spelled using “Heat Sink”. A heatsink is a block of non-ferrous metal that when cast or milled in a particular manner takes advantage of the thermodynamic properties of that material. By making one side a solid flat surface FIG. (1.4) and the opposing side having fins cut into that surface FIG. (1.2) we have the basic design and structure of a heatsink. Once an item is created, we have a heatsink that uses the principles of thermodynamics. As heat is applied to the flat surface of the heatsink and air is applied to the fins heat will travel from the flat surface to the fins in only one direction. Air moving thru the opposing fins pulls the heat to the fins and dissipates it into the air making the machined surface colder. Common embodiments of heatsinks are designed to protect Items producing high heat with a much cooler ambient temperature surrounding it. This invention addresses two new methods of use we can call out as such. The first new use for this item is to cool and or draw heat out of a living item. These items commonly might include human beings, animals, plants or any other kind of organic, biological item in this or other worlds (space). While the items listed in the prior statement can vary in size, this invention can be scaled to need by a simple calculation of. By making calculations involving side a the heat source side and b the fin side or cooling side we can always provide a minimum cooling factor of 3 to 1 ration. The square measurement unit of the fin surface must be three times minimum larger than the square measurement unit of the heat surface. A larger fin ratio to surface would result in a more powerful cooling and ability to pull heat. These results would appear to offer the following conclusion a unit with a 6 to 1 ratio would be twice as powerful as a unit with a 3 to 1 ratio. The common embodiment of a heatsink allows for a wide fluctuation in temperature between the heat surface and the cool ambient air. A Living item can have a range in the area of 20 degrees between the heat surface and the cool ambient air fin side making the above ratio a unique formula to create a new use for a heatsink. Commonly a heat sink is mounted to a like machined surface to allow for maximum contact with the heat source FIG. (1.3). As most but not all living items that might have need for a heat sink are soft and pliable the new shape and general geometry of the heat surface allows for a new way of making contact with a heat source. As this inventions heat source surface is ether slightly domed FIG. (2.1) to contour into a contour/pocket allowing the surface to have the heat removed to wrap around the heats source side having near 100% contact without a need to be mounted permanently. The other option for a heat source surface would be that of a slight dish shape FIG. (3.1) allowing for it to create a new use of wrapping around a positive contour or bump, again maintaining a near 100% contact without a need to be permanently mounted to draw heat out of living tissue. One key example of this unique new use would be for a woman having been treated and burned by radiation therapy to a breast for cancer. This would cup the breast and draw heat in a way that a commonly embodied heatsink could not do FIG. (3.1). The use of a plastic or rubber type material made as a ring FIG. (5.1) & FIG. (5.2) & FIG. (5.3) that will surround the exterior of this invention FIG. (4.1) & FIG. (4.2) will insulate the heat produced by holding the invention from affecting the efficiency of the unit's heat removal. For areas of a more cylindrical shape like a tube a plastic or rubber type material in a pliable sheet FIG. (6.1) can hold several of these new inventions or existing heatsinks side by side FIG. (6.2) allowing for the assembly to make contact in multiple places FIG. (7.1) while wrapping in a circular shape FIG. 7.3).By ether using the ring or pliable sheet as described above a commonly embodied heatsink might be retrofitted with one of these insolating handles, allowing for new uses without the need to mount it to a heat source making it more universal without the mounting limitations of commonly embodied heatsinks we see today.

1. The embodiments of the invention in which an exclusive property or privilege is claimed are defined as follows as a new use for an item commonly known as a heatsink having the benefits is to cool and or draw heat out of a living item. These items commonly might include human beings, animals, plants or any other kind of organic, biological item in this or other worlds (space)

2. The embodiments of the invention in which an exclusive property or privilege is claimed are defined as follows as a new use for an item commonly known as a heatsink having the benefits of use without the common requirement of the common heatsink in claim 1. That by providing a holding apparatus that mounts to the current invention exclusively and independent of its heat source the current invention can be held and moved independently without its thermodynamic traits being affected by the entity holding it. An example might be, but not exclusive as follows. The current invention might have a handle made of a plastic material that snaps over its exterior edges allowing it to be picked up in a human hand. The plastic ring would insolate any heat created by the hand from adding to the current inventions cooling abilities.

3. The embodiments of the invention in which an exclusive property or privilege is claimed are defined as follows as a new use for an item commonly known as a heatsink having the benefits of use without the common requirement of the common heatsink in claim 1. The use of a pliable and or flexible sheet of plastic material with properly sized and shaped holes. Multiple copies of common heatsinks and/or the current inventions can be shaped into the holes of the sheet allowing for the removal of heat from an arch shaped item and or tube shaped item. An example for description but not limited to embodiment or use would be the ability to wrap all or part of a human leg or arm.

4. The embodiments of the invention in which an exclusive property or privilege is claimed are defined as follows as a new use for an item commonly known as a heatsink having the benefits of use without the common requirement of the common heatsink in claim 1. To have a dish shaped side where a common heatsink described in claim 1 would be a flat surface allowing for the removal of heat out of a living item. These items commonly might include human beings, animals, plants or any other kind of organic, biological item in this or other worlds (space)

5. The embodiments of the invention in which an exclusive property or privilege is claimed are defined as follows as a new use for an item commonly known as a heatsink having the benefits of use without the common requirement of the common heatsink in claim 1. To have a dome shaped side where a common heatsink described in claim 1 would be a flat surface allowing for the removal of heat out of a living item. These items commonly might include human beings, animals, plants or any other kind of organic, biological item in this or other worlds (space)

6. The embodiments of the invention in which an exclusive property or privilege is claimed are defined as follows as a new use for an item commonly known as a heatsink having the benefits of use without the common requirement of the common heatsink in claim 1. The current invention having a dish shaped side where a common heatsink described in claim 1 would be a flat surface. The dish shape shall allow the current invention to be used to remove heat and/or resulting burns from a human breast that has been treated with radiation therapy for breast cancer to include the tissue area treated with radiation to the exterior skin in the path of radiation

6. The embodiments of the invention in which an exclusive property or privilege is claimed are defined as follows as a new use for an item commonly known as a heatsink having the benefits of use without the common requirement of the common heatsink in claim 1. The current invention having a dish shaped side where a common heatsink described in claim 1 would be a flat surface. The dome shape shall allow the current invention to be used to remove heat and/or resulting burns from a human all non (breast) locations that has been treated with radiation therapy for breast cancer to include the tissue area treated with radiation to the exterior skin in the path of radiation

7. The embodiments of the invention in which an exclusive property or privilege is claimed are defined as follows as a new use for an item commonly known as a heatsink having the benefits of use without the common requirement of the common heatsink in claim 1. The current invention having a dish shaped side where a common heatsink described in claim 1 would be a flat surface. The dome shape shall allow the current invention to be used to remove heat and/or resulting burns from human skin tissue. These burns are often refed to “sun burns”

8. The embodiments of the invention in which an exclusive property or privilege is claimed are defined as follows as a new use for an item commonly known as a heatsink having the benefits of use without the common requirement of the common heatsink in claim 1. The current invention having a dish shaped side where a common heatsink described in claim 1 would be a flat surface. The dome shape shall allow the current invention to be used to remove pain commonly known as a headache
